SEQUENCE LISTING (8 sequences)

SEQ ID NO: 1 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEHITSHMESEELNGAYKAIPVAQDLNAPSDWDSRGKDSYETSQLDDQSAEAHSHKQSRLYKRKANDESNEHSDVIDSQELSKVSREFHSHEFHSHEDMLVVDPKSKEEDKHLKFRISHELDSASSEVN

SEQ ID NO: 2 - protein, 170 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSK

SEQ ID NO: 3 - protein, 156 aa, Homo sapiens
MRIAVICFCLLGITCAIPVKQADSGSSEEKQLYNKYPDAVATWLNPDPSQKQNLLAPETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSK

SEQ ID NO: 4 - protein, 64 aa, Artificial Sequence
  Synthetic Peptide
MRIAVICFCLLGITCAGGGPGIPVKQADSGSSEEKGGGPGTPVVPTVDTYDGRGDSVVYGLRSK

SEQ ID NO: 5 - protein, 71 aa, Artificial Sequence
  Synthetic Peptide
MRIAVICFCLLGITCAGGGPGIPVKQADSGSSEEKGGGPGTPVVPTVDTYDGRGDSVVYGLRSKKAARGRR

SEQ ID NO: 6 - protein, 87 aa, Artificial Sequence
  Synthetic Peptide
MRIAVICFCLLGITCAGGGPGIPVKQADSGSSEEKGGGPGTPVVPTVDTYDGRGDSVVYGLRSKPAGAAGGPAGPAGPAGPAGPAGP

SEQ ID NO: 7 - DNA, 231 bp, Artificial Sequence
  Synthetic nucleic acid molecule
atgagaattgcagtgatttgcttttgcctcctaggcatcacctgtgccggcgggggccccggcataccagttaaacaggctgattctggaagttctgaggaaaagggcgggggccccggcactccagttgtccccacagtagacacatatgatggccgaggtgatagtgtggtttatggactgaggtcaaaaaaagctgctcgcggccgccgcgctgctcgcggccgccgc

SEQ ID NO: 8 - protein, 76 aa, Artificial Sequence
  Synthetic Peptide
MRIAVICFCLLGITCAGGGPGIPVKQADSGSSEEKGGPGTPVVPTVDTYDGRGDSVVYGLRSKKAARGRRAARGRR

(19) United States
(12) Patent Application Publication
(10) Pub. No.: US 2004/0034193 A1
(43) Pub. Date: Feb. 19, 2004

(54) Biosynthetic oncolytic molecules and uses therefor

(76) Inventors: Samy Ashkar, Boston, MA (US); Sherry Hikita, Santa Barbara, CA (US); Ghassan Dehni, Boston, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 10/220,107

(22) PCT Filed: Jun. 13, 2001
(86) PCT No.: PCT/US01/19239

(51) Int. Cl. A61K 38/17; C07K 14/47

(52) U.S. Cl. 530/350; 514/12

(57) ABSTRACT

Novel biosynthetic oncolytic molecules are provided. The biosynthetic oncolytic molecules include functional domains derived from osteopontin. Preferred biosynthetic oncolytic molecules include an apoptotic domain derived from osteopontin. The oncolytic molecules of the present invention are capable of promoting cellular apoptosis. Accordingly, therapeutic uses are disclosed which feature the biosynthetic oncolytic molecules of the present invention.

### BACKGROUND OF THE INVENTION

Cellular apoptosis, or programmed cell death, is a mechanism by which distinct subsets of cells are deleted during embryonic development and in normal cell turnover in tissues. Apoptosis is also initiated following various forms of cellular injury including viral infection, exposure to toxic agents, and irradiation. The balance between cell proliferation and/or survival, and cell death is an important component of normal physiology as well as the pathogenesis of diseases characterized by deregulated growth control, such as cancer.

Osteopontin is a ubiquitous extracellular matrix phosphoprotein that functions in cell adhesion and migration. Osteopontin may also initiate intracellular signal transduction pathways via two types of receptors, the αvβ3 integrins and the proteoglycan CD44. Osteopontin has been shown to be an important mediator of the cellular response to oxidative stress, where it exerts antioxidant and anti-apoptotic effects. Moreover, osteopontin is capable of inhibiting apoptosis in several cell types that, recognize osteopontin (Weber, G. F. et al. (1997) Proc. Assoc. Am. Phys., 109:1-9). The expression of osteopontin is also associated with pathological states including atherosclerosis and tumorigenesis and metastasis (Oates, A. J. et al. (1997) Invasion Metast., 17:1-15).

### SUMMARY OF THE INVENTION

Given the observed indications of a role for osteopontin in the regulation of cellular responses to stress, there exists a need for a more precise understanding of the mechanism by which osteopontin affects cellular viability. Inhibition of apoptosis by osteopontin requires the coordinated ligation of (and signaling through) both CD44 and αvβ3 integrin. The present invention is based, at least in part, on the surprising discovery that misligation of αvβ3 integrin and CD44 results in apoptosis. In particular, induction of apoptosis results from engagement of αvβ3 integrin and CD44 by an N-terminal osteopontin which is sufficient to engage but not activate αvβ3 integrin and which both engages and activates CD44. Engagement of αvβ3 blocks any signaling (e.g., MAPK signaling) through that receptor. Engagement of CD44 activates JNK signaling, which in the absence of MAPK signaling, results in activation of apoptosis. Based on a detailed understanding of the functional domains of osteopontin and an understanding of the role this multifunctional cytolcine plays in the regulation of cellular apoptosis, the present invention provides biosynthetic molecules which mimic distinct functions of osteopontin for use in a variety of therapeutic applications, in particular, in the treatment of cancer and inflammatory conditions such as arthritis. In particular, the biosynthetic molecules of the present invention are useful in the elimination of abnormal or unwanted cells that express at least an integrin receptor and/or that co-express both an integrin and a CD44 receptor.

### BRIEF DESCRIPTION OF THE DRAWINGS

Other features and advantages of the invention will be apparent from the following detailed description, and from the claims.

FIG. 1A depicts the amino acid sequences of human osteopontin-B (OPN-b) (SEQ ID NO: 1), a preferred splice variant of the human osteopontin gene.

FIGS. 1B-C depicts the amino acid sequences of OPN-a/nt (SEQ ID NO: 2) and OPN-b/nt (SEQ ID NO: 3), which represent truncated derivatives of human osteopontin-A and osteopontin-B, respectively, that induce apoptosis.

FIG. 1D depicts a first generation biosynthetic oncolytic molecule termed “oncolysin N” (SEQ ID NO: 4).

FIG. 2A depicts the amino acid sequences of two second generation biosynthetic oncolytic molecules oncolysin 1 (SEQ ID NO: 5) and oncolysin 2 (SEQ ID NO: 6) derived from oncolysin N.

FIG. 2B depicts the nucleotide and amino acid sequences of the second generation biosynthetic oncolytic molecule, oncolysin 3 (SEQ ID NOs: 7 and 8, respectively).

FIG. 3 depicts an alteration in the signal transduction pathway in cells infected with oncolysin 1/Sophin C as compared to control cells. The bar graph quantitates the decreased SHP-1 protein expression and increased PI-3 kinase expression.

FIG. 4 depicts the effect of oncolysin 1-infection on tumor volume in an experimental animal tumor model.

FIG. 5 depicts the effect of oncolysin 1 administration in different tumor models and at different doses.

### DETAILED DESCRIPTION OF THE INVENTION

The present invention is based, at least in part, on the elucidation of a new function for osteopontin as a modulator of cellular apoptosis. In particular, it has been discovered that osteopontin comprises a domain which when isolated from osteopontin has the capacity to induce cellular apoptosis. Binding of this apoptosis fragment mis-ligates osteopontin receptors resulting in cellular apoptosis. In particular this fragment binds CD44vand αvβ3 integrin when co-expressed on cells. As the co-expression is extremely rare under normal circumstances, proteins which include this apoptotic fragment can be exploited to destroy abnormal cells which do co-express these receptors, including several metastatic cells and hyperactivated macrophages such as those involved in arthritis.

Based on the discovery of an oncolytic function of osteopontin, and in particular, the discovery of an apoptotic domain, the present invention features biosynthetic molecules which are modeled after the osteopontin derived apoptotic fragment. The biosynthetic molecules are useful in regulating cellular growth processes, as well as in promoting apoptosis. Accordingly, in one embodiment, the present invention features biosynthetic oncolytic molecules which include an apoptotic component and a biomodular component, forming a molecule which promotes apoptosis. The term “biosynthetic molecule” includes molecules which are built or synthesized by a combination or union of components or elements that are simpler than the elements of the naturally occurring protein and accordingly, have only selected activities of the naturally occurring molecule. A biosynthetic molecule of the present invention is made or built by the hand of man (including automated processes) and accordingly, is distinguishable from a naturally-occurring molecule which is results from a naturally-occurring biological process. Alternatively, an organism can be used to produce a biosynthetic molecule of the present invention, provided that at least at one step in the synthesis, there is the intervention of man.

The term “oncolytic or “oncolytic molecule” includes molecules which have a modulatory or regulatory activity which is normally associated with an apoptotic response in an organism, for example, higher animals and humans. An activity (e.g., a biological or functional activity) associated with an apoptotic response can be any activity associated with the induction of programmed cell death in response to developmental signals, adverse growth conditions, viral infection, cellular injury, or disease. The term “activity”, “biological activity” or “functional activity”, refers to an activity exerted by a molecule of the invention (e.g., a biosynthetic molecule or a protein, polypeptide or nucleic acid molecule) as determined in vivo, or in vitro, according to standard techniques.

The term “apoptotic response” includes any response associated with the induction of programmed cell death including, but not limited to chromatin condensation and fragmentation, decreased cell viability, and cell lysis. The phrase “modulates an apoptotic response” or “modulator of an apoptotic response” includes upregulation, enhancing or increasing an apoptotic response, as defined herein. The phrase “modulates an apoptotic response” or “modulator of an apoptotic response” also includes downregulation, inhibition or decreasing an apoptotic response as defined herein.

The present invention further features biosynthetic oncolytic molecules which include an apoptotic component. The term “apoptotic component” (also referred to herein as an “apoptotic domain” or “pro-apoptotic domain”) includes a piece or constituent of a molecule which is smaller than the molecule of which it is a part, which functions to promote apoptosis of a cell. As defined herein, an apoptotic component includes a component which is capable at ligating an integrin (e.g., αvβ3) and CD44 (e.g., CD44V) expressed on a cell surface, resulting in signaling through CD44 (e.g., activation of the JNK signaling pathway) and blocking of integrin signaling (e.g., blocking binding of any other ligand capable of activating MAPK signaling). A molecule which includes an apoptotic component, for example, is capable of causing a viable cell to undergo apoptosis in the presence of the apoptotic component as compared to the same cell in the absence of the apoptotic component. A preferred apoptotic component or pro-apoptotic domain comprises amino acids 147-170 of the human osteopontin sequence set forth as SEQ ID NO: 1. Alternatively, an apoptotic component or pro-apoptotic domain contains 0-5, 5-10, 10-15 or 15-20 consecutive amino acid residues N terminal or C terminal to amino acids 147-170 of SEQ ID NO: 1 and retains at least 60%, preferably at least 70%, more preferably at least 80%, and even more preferably 90-95% of the apoptotic activity of the domain consisting of amino acids 147-170 of SEQ ID NO: 1 (e.g., as determined in any art recognized in vitro apoptosis assay, either when assayed alone or in the context of a biosynthetic molecule as defined herein.) In yet another embodiment, the apoptotic component or pro-apoptotic domain contains fewer that the 24 amino acid residues from 147-170 of SEQ ID NO: 1 (e.g., contains only 15, 16, 17,18, 19, 20, 21, 22 or 23 consecutive amino acid residues of the sequence from 147 to 170 of SEQ ID NO: 1 yet retains at least 60%, preferably at least 70%, more preferably at least 80%, and even more preferably 90-95% of the apoptotic activity of the domain consisting of amino acids 147-170 of SEQ ID NO: 1. In yet another embodiment, the apoptotic component or pro-apoptotic domain has 1, 2, 3, 4, 5, 6, 7, 8, 9 or 10 amino acid residues substituted yet retains at least 60%, preferably at least 70%, more preferably at least 80%, and even more preferably 90-95% of the apoptotic activity of the domain consisting of amino acids 147-170 of SEQ ID NO: 1.

In addition to an apoptotic component, the biosynthetic molecules of the present invention can include a biomodular component. The term “biomodular component” includes a piece or constituent of a molecule which is smaller than the molecule of which it is a part, which has either a biological function which is distinct from that of the apoptotic component or has a biological structure which is distinct from that of the apoptotic component. A biomodular component is a piece or constituent that either is not found in a naturally-occurring molecule which includes an apoptotic component or is not found in the same proximal relation to an apoptotic component as it exists within a naturally-occurring molecule. In one embodiment, a biomodular component is a polypeptide. Polypeptide biomodular components of the present invention include, but are not limited to signal peptides, a linker domain, and a golgi processing domain.

The term “signal peptide” or “signal sequence” refers to a peptide containing about 20 amino acids which occurs at the N-terminus of secretory and integral membrane proteins and which contains a large number of hydrophobic amino acid residues. For example, a signal sequence contains at least about 14-28 amino acid residues, preferably about 16-26 amino acid residues, more preferably about 18-24 amino acid residues, and more preferably about 20-22 amino acid residues, and has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence from the endoplasmic reticulum of a cell to the golgi apparatus and ultimately to a lipid bilayer (e.g., for secretion). A preferred signal sequence is derived from human osteopontin (e.g., comprises amino acids 1-16 of the human osteopontin sequence set forth as SEQ ID NO: 1.

The term “linker” includes a domain which, when included within a protein, polypeptide, or biosynthetic molecule of the present invention, functions to minimize globular folding, separate modular proteins into distinct functional domains, and maintain functionality of the protein, peptide, or biosynthetic molecule.

The term “golgi processing domain” includes a domain which, when included within a protein, polypeptide, or biosynthetic molecule of the present invention, functions to confer upon the molecule the ability to be secreted from the cell via transport through the endoplasmic reticulum and golgi apparatus, and/or modified within the endoplasmic reticulum and golgi apparatus, e.g., via the addition of carbohydrate residues. A preferred golgi processing domain is derived from human osteopontin (e.g., includes amino acids 17-30 of the sequence set forth as SEQ ID NO: 1). Additional exemplary biomodular components include, for example, heparin binding domains and or collagen binding domains. As used herein, the term “heparin binding domain” includes a component which facilitates binding of a biosynthetic molecule to extracellular matrix components, e.g., with heparin in the extracellular matrix surrounding a target cell, to stabilize the interaction of the biosynthetic molecule with the target cell. A “heparin binding domain” includes at least one, preferably two, more preferably three, four, five or six “heparin binding motifs” having the formula arg-xaa-basic residue-basic residue, preferably, arg-xaa-(arg or lys)-(arg or lys). Exemplary heparin binding motifs include RXRR, RXKK, RXRK and RXKR. Consecutive heparin binding motifs are preferably separated by any two amino acids, ie., are separated by xaa-xaa. Thus a preferred heparin binding domain has the formula (R—X—R/K)−(X—X—R—X—R/K)ₙ, where n=1, preferably 2, more preferably 3 or 4. In a preferred embodiment, n=2. (Additional consecutive heparin binding motifs can be added but, ultimately, will decrease rather than increase the apoptotic effectiveness of the biosynthetic oncolytic molecule.) A particularly preferred heparin binding domain has the amino acid sequence RSKKAARGRR (amino acids 62 to 71 of SEQ ID NO: 6). Another particularly preferred heparin binding domain has the amino acid sequence RSKKAARGRRAARGRR (amino acids 62 to 77 of SEQ ID NO: 8)

As used herein, the term “collagen binding domain” includes a component which facilitates binding of a biosynthetic molecule to extracellular matrix components, e.g., with collagen in the extracellular matrix surrounding a target cell, to stabilize the interaction of the biosynthetic molecule with the target cell. A particularly preferred collagen binding domain has the amino acid sequence PAGAAGGPAGPAGPAGPAGPAGP (amino acids 65 to 87 of SEQ ID NO: 6).

Accordingly, a biosynthetic molecule of the present invention is formed by the combination of at least an apoptotic domain and a biomodular component. The term “formed” or “forming” includes the bringing together of at least two components into a structural and/or functional association. For example, a recombinant nucleic acid molecule can be formed by the bringing together of at least two nucleic acid components. Alternatively, a recombinant protein can be formed by the bringing together of at least two protein components. Moreover, a composition can be formed by the bringing together of at least two compositions.

In a preferred embodiment, the present invention features biosynthetic molecules which include an apoptotic component which is derived from osteopontin. A component “derived from”, for example, osteopontin, includes a component which has certain features which originate from osteopontin and are recognizable as such, but which is not identical to osteopontin. Preferably, an apoptotic component has sufficient sequence information to bind integrin (e.g., αvβ3 integrin) but lacks sufficient sequence information to signal via integrin (e.g., via αvβ3 integrin). In one embodiment, an apoptotic component is a polypeptide which is derived from osteopontin. Accordingly, the apoptotic component has features of osteopontin (e.g., functions to promote apoptosis) but is not identical to osteopontin. In one embodiment, an apoptotic component includes a polypeptide which has at least 50% identity to an apoptotic domain of osteopontin. In yet another embodiment, an apoptotic component is at least 55%, 65%, 70%, 75%, 80%, 85%, 90%, 95%, or more identical to an apoptotic domain of osteopontin. In yet another embodiment, an apoptotic component includes an amino acid sequence consisting of amino acids 147-170 of human osteopontin-B (SEQ ID NO: 1). In another embodiment, an apoptotic component includes a polypeptide which is at least 55%, 65%, 70%, 75%, 80%, 85%, 90%, 95%, or more identical to about amino acids 147-170 of human osteopontin-B (SEQ ID NO: 1). In another embodiment, an apoptotic component includes a polypeptide which is at least 5-50 amino acids in length. In another embodiment, an apoptotic component includes a polypeptide which is between 10-45, 15-40, or 20-30, or 21, 22, 23, 24, 25, 26, 27, 28, or 29 amino acids in length. In another embodiment, an apoptotic component includes a polypeptide which is greater than 50 amino acids in length.

Another embodiment of the present invention features biosynthetic molecules which include an apoptotic component having an amino acid sequence sufficiently homologous to the apoptotic domain of human osteopontin (e.g., amino acids 147-170 of SEQ ID NO: 1). The term “sufficiently homologous” includes a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share at least 40%, preferably 50%, more preferably 60%, 70%, 80% or 90% identity and share a common functional activity are defined herein as sufficiently homologous. In a preferred embodiment, an apoptotic component retains an apoptotic activity, preferably an apoptotic activity of osteopontin. In another embodiment, a molecule retains an oncolytic activity. The present invention further features isolated nucleic acid molecules which encode the biosynthetic oncolytic molecules of the present invention. In one embodiment, an isolated nucleic acid molecule of the present invention includes a nucleic acid sequence which encodes an apoptotic domain. In another embodiment, an isolated nucleic acid molecule of the present invention includes a nucleic acid sequence which encodes a biomodulatory domain.

Various aspects of the invention are described in further detail in the following subsections:

I. Isolated Nucleic Acid Molecules

One aspect of the invention pertains to isolated nucleic acid molecules that encode biosynthetic molecules or portions thereof (e.g., a portion encoding a biomodular domain, for example, an apoptotic domain). The term “nucleic acid molecule” includes DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

In another preferred embodiment, an isolated nucleic acid molecule of the invention comprises a nucleic acid molecule encodes at least an apoptotic domain of osteopontin (e.g., amino acids 147 to 170 of SEQ ID NO: 1). Preferably, an isolated nucleic acid molecules encodes the biosynthetic molecules of any of SEQ ID NO: 4, SEQ ID NO: 5, SEQ ID NO: 6 and SEQ ID NO: 8. An exemplary nucleic acid is set forth as SEQ ID NO: 7.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed with the NBLAST program, score=100, wordlength=12 to obtain nucleotide sequences homologous to oncostatin nucleic acid. molecules of the invention. BLAST protein searches can be performed with the XBLAST program, score=50, wordlength=3 to obtain amino acid sequences homologous to oncostatin protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

A nucleic acid of the invention, or portion thereof, can be amplified using cDNA, MRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides can be prepared by standard synthetic techniques, e.g., using an automated DNA synthesizer. Probes/primers for use in the present invention typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense sequence encoding SEQ ID NO: 1.

A nucleic acid fragment encoding a “biologically active” portion of a biosynthetic molecule of the present invention can be prepared by isolating a portion of a nucleic acid molecule which encodes a polypeptide having a biological activity of the naturally-occurring protein from which the portion was derived, expressing the encoded portion of the naturally-occurring protein (e.g., by recombinant expression in vitro) and assessing the activity of the encoded portion of the naturally-occurring protein. As used herein, a “naturally-occurring” nucleic acid molecule refers to an RNA or DNA molecule having a nucleotide sequence that occurs in nature (e.g., encodes a natural protein).

The invention further encompasses nucleic acid molecules that differ due to degeneracy of the genetic code but encode the same biosynthetic molecules (e.g. encoding a protein having the amino acid sequence shown in SEQ ID NO: 4, SEQ ID NO: 5, SEQ ID NO: 6 or SEQ ID NO: 8).

In addition to the biosynthetic molecule amino acid sequences of the present invention, the skilled artisan will appreciate that changes can be introduced by mutation into the nucleotide sequences encoding such amino acid sequences thereby leading to changes in the amino acid sequence of the encoded biosynthetic molecule without altering function. For example, nucleotide substitutions leading to amino acid substitutions (particularly conservative amino acid substitutions) at “non-essential” amino acid residues can be made in the encoding nucleic acid sequence. A “non-essential” amino acid residue is a residue that can be altered from the sequence (e.g., amino acids 147 to 170 of SEQ ID NO: 1) without altering the biological activity, whereas an “essential” amino acid residue is required for biological activity. For example, amino acid residues that are conserved among proteins or domains of proteins from different species are predicted to be particularly unamenable to alteration. Accordingly, another aspect of the invention pertains to biosynthetic molecule-encoding nucleic acid molecules that encode changes in amino acid residues that are not essential for activity. The encoded products may differ in amino acid sequence from, for example, from amino acids 147 to 170 of SEQ ID NO: 1, yet retain biological activity. In one embodiment, an isolated nucleic acid molecule comprises a nucleotide sequence encoding a protein which is at least about 60% homologous to amino acids 147 to 170 of SEQ ID NO: 2. Preferably, the protein encoded by the nucleic acid molecule is at least about 65-70% homologous to amino acids 147 to 170 of SEQ ID NO: 1, more preferably at least about 75-80% homologous to amino acids 147 to 170 of SEQ ID NO: 1, even more preferably at least about 85-90% homologous to amino acids 147 to 170 of SEQ ID NO: 1, and most preferably at least about 95% homologous to amino acids 147 to 170 of SEQ ID NO: 1.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis of a nucleic acid encoding SEQ ID NO: 1, the newly-encoded protein can be expressed recombinantly and the activity of the protein can be determined.

II. Isolated Biosynthetic Molecules

One aspect of the invention pertains to isolated biosynthetic molecules and portions thereof. In one embodiment, the biosynthetic molecules of the present invention are produced by recombinant DNA techniques. Alternative to recombinant expression, a biosynthetic molecule can be synthesized chemically using standard peptide synthesis techniques.

An “isolated” or “purified” biosynthetic molecule is substantially free of cellular material or other contaminating proteins from the cell or tissue source from which the molecule is derived, or substantially free from chemical precursors or other chemicals when chemically synthesized. The language “substantially free of cellular material” includes preparations in which the recombinant molecule is separated from cellular components of the cells from which it is isolated or recombinantly produced. In one embodiment, the language “substantially free of cellular material” includes preparations having less than about 30% (by dry weight) of non-biosynthetic molecule (also referred to herein as a “contaminating material”), more preferably less than about 20% of contaminating material, still more preferably less than about 10% of contaminating material, and most preferably less than about 5% contaminating material. When the biosynthetic molecules of the present invention are recombinantly produced, it is also preferably substantially free of culture medium, i.e., culture medium represents less than about 20%, more preferably less than about 10%, and most preferably less than about 5% of the volume of the preparation.

The language “substantially free of chemical precursors or other chemicals” includes preparations in which the biosynthetic molecule is separated from chemical precursors or other chemicals which are involved in the synthesis of the molecule. In one embodiment, the language “substantially free of chemical precursors or other chemicals” includes preparations having less than about 30% (by dry weight) of chemical precursors or contaminating chemicals, more preferably less than about 20% chemical precursors or contaminating chemicals, still more preferably less than about 10% chemical precursors or contaminating chemcals, and most preferably less than about 5% chemical precursors or contaminating chemicals.

Biologically active portions of a biosynthetic molecule of the present invention include molecules sufficiently homologous to or derived from the biosynthetic molecules of the present invention, e.g., the amino acid sequence shown in SEQ ID NO: 1, which include less amino acids than the fill length polypeptide, and exhibit at least one activity of the full-length polypeptide. Typically, biologically active portions comprise a domain or motif with at least one activity of the full-length polypeptide. A biologically active portion can be a polypeptide which is, for example, 10, 25, 50, 100 or more amino acids in length.

The invention also provides chimeric or fusion proteins. The term “chimeric protein” or “fusion protein” includes a first polypeptide (e.g., an osteopontin-derived polypeptide) operatively linked to a second polypeptide (e.g., a non-osteopontin-derived polypeptide). An “osteopontin-derived polypeptide” refers to a polypeptide having an amino acid sequence derived from osteopontin, whereas a “non-osteopontin-derived polypeptide” refers to a polypeptide having an amino acid sequence corresponding to a protein which is not substantially homologous to osteopontin. Within a fusion protein the first polypeptide can correspond to all or a portion of osteopontin. In a preferred embodiment, a fusion protein comprises at least one biologically active portion of osteopontin. In another preferred embodiment, a fusion protein comprises at least two biologically active portions of osteopontin. Within the fusion protein, the term “operatively linked” is intended to indicate that the first polypeptide and the second polypeptide are fused in-frame to each other. The first polypeptide can be fused to the N-terminus or C-terminus of the second polypeptide.

For example, in one embodiment, the fusion protein is a GST-fusion protein in which the polypeptide sequences of interest (e.g., apoptotic domain sequences) are fused to the C-terminus of the GST sequences. Such fusion proteins can facilitate the purification of recombinant proteins.

In another embodiment, the fusion protein contains a heterologous signal sequence at its N-terminus. For example, the native osteopontin signal sequence (i.e., about amino acids 1 to 16 of SEQ ID NO: 1) can be removed and replaced with a signal sequence from another protein. In certain host cells (e.g., mammalian host cells), expression and/or secretion of fusion proteins can be increased through use of a heterologous signal sequence.

In yet another embodiment, the fusion protein is an immunoglobulin fusion protein in which the sequences of interest (e.g., apoptotic domain sequences) are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon et al. (1989) Nature 337:525-531 and Capon U.S. Pat. Nos. 5,116,964 and 5,428,130 [CD4-IgG1 constructs]; Linsley et al. (1991) J. Exp. Med. 173:721-730 [a CD28-IgG1 construct and a B7-1-IgG1 construct]; and Linsley, et al (1991) J. Exp. Med. 174:561-569 and U.S. Pat. No. 5,434,131 [a CTLA4-IgG1]).

The immunoglobulin fusion proteins of the invention can be incorporated into pharmaceutical compositions and administered to a subject and may be useful therapeutically for the modulation of cellular apoptosis. Moreover, the immunoglobulin fusion proteins of the invention can be used as immunogens to produce antibodies in a subject, to purify ligands and in screening assays.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A nucleic acid encoding a domain of interest (e.g., apoptotic domain sequences) can be cloned into such an expression vector such that the fusion moiety is linked in-frame to the domain of interest.

III. Recombinant Expression Vectors and Host Cells

Another aspect of the invention pertains to vectors, preferably expression vectors, containing a nucleic acid encoding a domain of interest (e.g., apoptotic domain sequences). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” means that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression in prokaryotic or eukaryotic cells. For example, recombinant proteins can be expressed in bacterial cells such as E. coli, insect cells (using baculovirus expression vectors) yeast cells or mammalian cells. Suitable host cells are discussed further in Goeddel, Gene Expression Technology. Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, recombinant proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and inununoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to oncostatin mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, oncostatin protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning. A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding recombinant proteins or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) recombinant protein. Accordingly, the invention further provides methods for producing recombinant protein using the host cells of the invention. In one embodiment, the method comprises culturing the host cell of invention (into which a recombinant expression vector encoding recombinant protein has been introduced) in a suitable medium such that the recombinant protein is produced. In another embodiment, the method further comprises isolating the recombinant protein from the medium or the host cell.

IV. Pharmaceutical Compositions

The nucleic acid molecules, proteins, and biosynthetic molecules (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a oncostatin protein or anti-oncostatin antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The present invention provides for both prophylactic and therapeutic methods of treating subjects (e.g., human subjects). In one aspect, the invention provides a method for preventing in a subject prophylactically. Administration of a agent prophylactically can occur prior to the manifestation of symptoms of an undesired disease or disorder, such that the disease or disorder is prevented or, alternatively, delayed in its progression. The prophylactic methods of the present invention can be carried out in a similar manner to therapeutic methods described herein, although dosage and treatment regimes may differ.

Another aspect of the invention pertains to methods for treating a subject therapeutically. In one embodiment, the present invention includes methods of modulating an apoptotic response. In particular, modulation of an apoptotic response includes, but is not limited to, modulation of cellular chromatin structure, modulation of cell viability, or modulation of cell lysis. A preferred embodiment of the invention involves modulation of apoptosis, in particular, promotion of programmed cell death. Accordingly, the present method has therapeutic utility in eliminating abnormal or unwanted cells. Such a modulatory method is particularly useful in diseases such as cancer, and in inflammatory diseases characterized by the hyperactivation of macrophages, e.g. arthritis.

The modulatory method of the invention involves contacting a cell with an agent that modulates one or more of the activities associated with an apoptotic response. In a preferred embodiment, a cell contacted with a biosynthetic oncolytic molecule of the present invention is present within a subject. Contacting cells within a subject can be accomplished by direct administration of the biosynthetic molecule or by retroviral delivery of the molecule as exemplified in Examples 3-5, or by any art-recognized means for introducing or expressing polypeptides within a subject.

The present invention is further illustrated by the following Example which in no way should be construed as further limiting. The entire contents of all of the references (including literature references, issued patents, and published patent applications) cited throughout this application are hereby expressly incorporated by reference.

### EXEMPLIFICATION

### Example 1

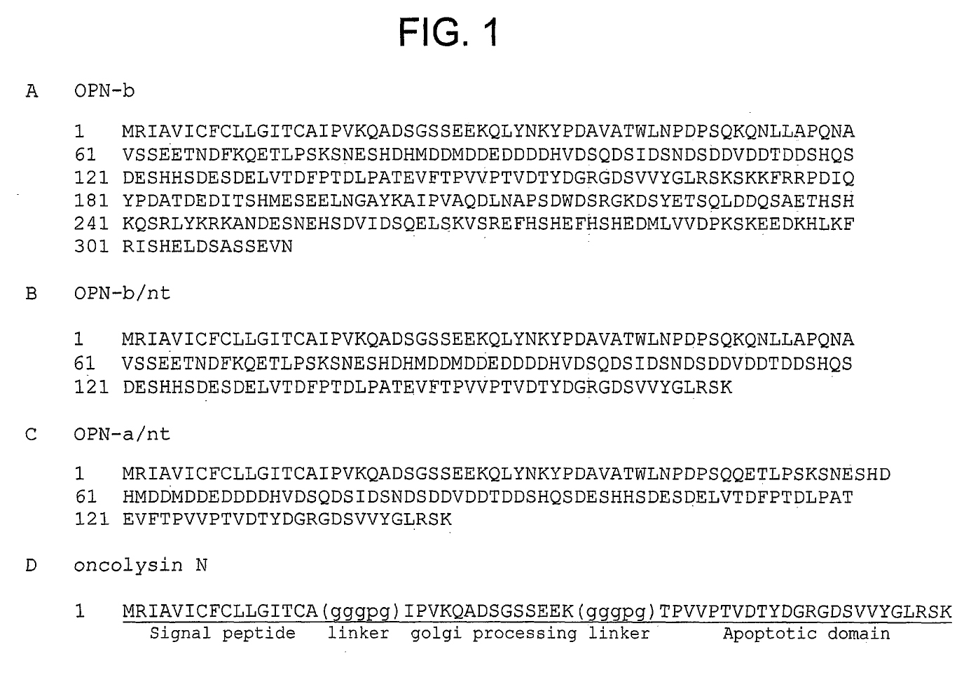

A first generation osteopontin-derived biosynthetic molecule, oncolysin N, was engineered based on the isolation of a domain of osteopontin sufficient to impart pro-apoptotic activity when isolated away from the naturally-occurring osteopontin polypeptide. In particular, the oncolysin N molecule was designed to include the following domains: (1) a signal sequence (i.e., signal peptide), derived in this instance from the native osteopontin amino acid sequence (i.e., amino acids 1-16 of the human osteopontin-B amino acid sequence set forth as SEQ ID NO: 1); (2) a golgi processing domain derived from the native osteopontin amino acid sequence (ie., amino acids 17-30 of the human osteopontin-B amino acid sequence set forth as SEQ ID NO: 1); (3) a pro-apoptotic domain comprising contiguous amino acid residues of human osteopontin-B sufficient to induce apoptosis (i.e., amino acids 147-170 of the human osteopontin-B amino acid sequence set forth as SEQ ID NO: 1) yet lacking additional osteopontin-B sequences which are unnecessary for apoptotic activity, or alternatively decrease pro-apoptotic activity, of the biosynthetic molecule; and (4) two linker domains, a first linker domain operably linking the signal sequence to the golgi processing domain, and a second linker domain operably linking the golgi processing domain to the pro-apoptotic domain. The signal sequence and golgi processing domain optimize synthesis, processing through the golgi and secretion of the biosynthetic oncolysin N molecule. The linker domains force independent folding of the functional domains. The signal sequence of oncolysin N is cleaved between gly17 and gly 18 of SEQ ID NO: 4 with the mature polypeptide having the N-terminal sequence GGPGIPVK (corresponding to amino acids 18-25 of SEQ ID NO: 4). The oncolysin N molecule, termed a “first generation” biosynthetic oncolytic molecule, has the ability to modulate apoptotic responses, in particular, the ability to promote cellular apoptosis. In apoptosis assays, native osteopontin-B has no apoptotic activity, whereas certain N-terminal osteopontin bioactive fragments have the ability to at least partially induce apoptosis (i.e., the N-terminal osteopontin a and osteopontin b sequences, OPN-a/nt and OPN-b/nt, set forth in FIGS. 1B-C and SEQ ID NOs: 2-3, respectively). The biosynthetic oncolysin N molecule was likewise at least partially effective at inducing apoptosis. Induction of apoptotic activity can be performed according to any one of a number of art-recognized assays. An exemplary assay is set forth below, i.e., the induction of apoptosis by a osteopontin-derived biosynthetic molecule, oncolysin N, in a metastatic tumor cell line.

Cells are grown in culture and treated with varying doses of exogenous oncolysin N. Apoptosis is determined by flow cytometric analysis according to the uptake of propidium iodide. Cells are harvested in phosphate buffered saline containing 5 mM EDTA and fixed in 50% ethanol for 30 minutes. RNA is removed by treatment with 40 μM RNAse A for 30 minutes at room temperature, and cells are incubated with 100 μg/mL propidium iodide in phosphate buffered saline containing 5 mM EDTA. DNA cleavage in apoptotic cells is assessed by flow cytometric analysis, as cells containing hypodiploid nuclei bind less propidium iodide than intact nuclei.

Cellular apoptosis can also be determined using standard criteria in the art such as nuclear condensation, chromatin fragmentation, and viability as assessed by Trypan blue exclusion.

### Example 2

This example describes the engineering of two “second generation” biosynthetic oncolytic molecules based on the structure and activity of oncolysin N. In particular, two second generation biosynthetic oncolytic molecules were generated from oncolysin N, oncolysin 1 (also referred to herein as “Sophin C”) and oncolysin 2.

To generate oncolysin 2, a synthetic collagen binding domain was engineered at the C terminus of oncolysin N. The amino acid sequence of oncolysin 2 is set forth as SEQ ID NO: 6. Cellular apoptosis assays are as described in example 1. Oncolysin 2 was more effective at promoting apoptosis than oncolysin N.

To generate oncolysin 1/Sophin C, a synthetic heparin binding domain was engineered at the C terminus of oncolysin N. A “heparin binding domain” includes at least one, preferably two, more preferably three, four, five or six heparin binding motifs having the formula arg-xaa-basic residue-basic residue, preferably, arg-xaa-(arg or lys)-(arg or lys). Exemplary heparin binding motifs include RXRR, RXKK, RXRK and RXKR. Consecutive heparin binding motifs are preferably separated by any two amino acids, i.e., are separated by xaa-xaa. Thus a preferred heparin binding domain has the formula (R—X—R/K)−(X—X—R—X—R/K)ₙ, where n=1, preferably 2, more preferably 3 or 4. In a preferred embodiment, n=3. (Additional consecutive heparin binding motifs can be added but, ultimately, will decrease rather than increase the apoptotic effectiveness of the biosynthetic oncolytic molecule.) The addition of the heparin binding domain was found to dramatically increase apoptotic activity. The amino acid sequence of oncolysin 1/Sophin C (having the two heparin binding motifs, RSKK and RGRR) is set forth as SEQ ID NO: 5. Mechanistic analysis demonstrated that including additional heparin binding motifs enhances misligation of the integrin receptor on a tumor cell's surface with a second receptor, for example, CD44 or a growth factor receptor (e.g., a growth factor receptor such as an EGF—R or hbGF—R). An exemplary misligated receptor is her-2, which is expressed by breast cancer cells, making the hereindescribed biosynthetic oncolytic molecules effective against breast cancer cells.

### Example 3

This example demonstrates the production of a retroviral expression vector allowing for the stable induction of high levels of oncolysin 1/Sophin C expression in mammalian hosts, both in vitro and in vivo.

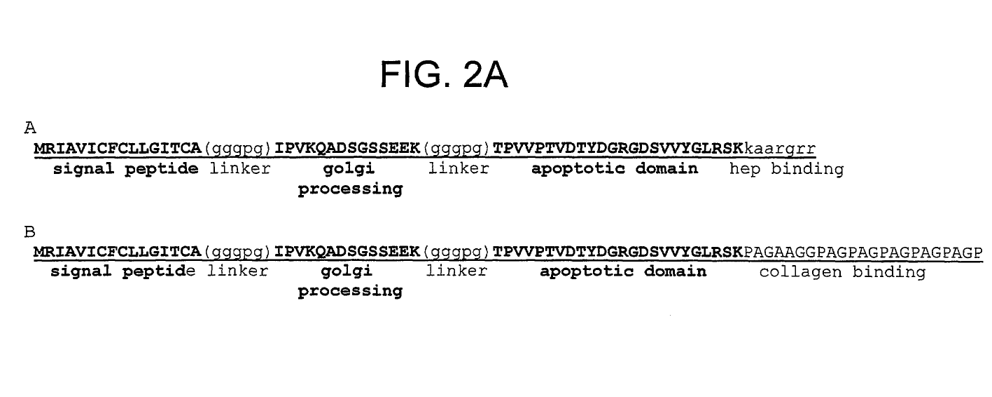

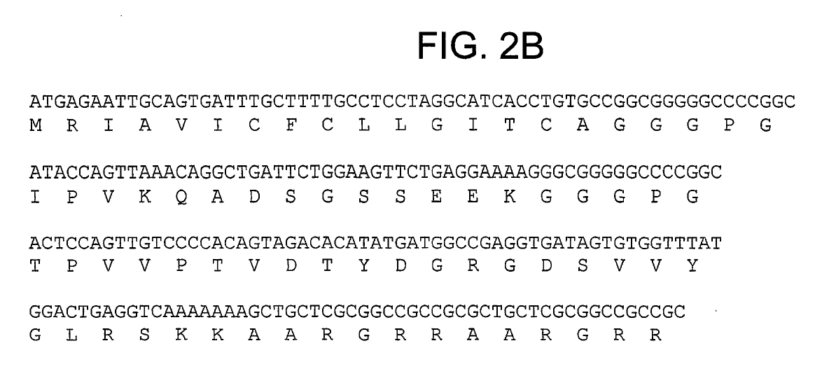

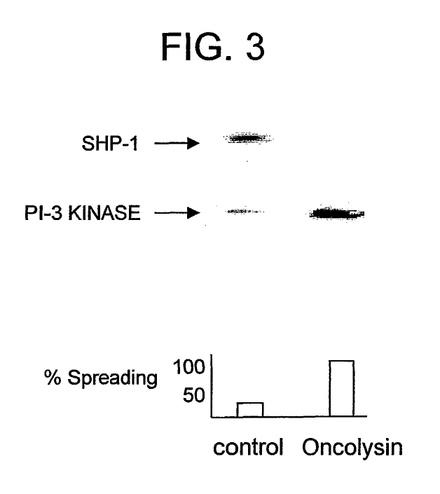

Oncolysin was cloned into a 9 kb retroviral Tet—On expression vector. These vectors are designed for high level stable expression in mammalian hosts. The retroviral Tet-inducible vector produces infectious, replication—incompetent retrovirus that can be used to introduce a gene of interest into a wide variety of mammalian cell types in vitro and in vivo. The highly efficient transduction machinery of retroviruses can stably integrate the cloned gene into the host genome of nearly all mitotically active cells. The tetracycline (Tc) controlled transactivator and the reverse Tc controlled tranactivator (rtTA) are expressed from the same integrated retroviral construct containing the gene of interest. RtTA binds the TRE and activates transcription in the presence of Doxycycline. The gene of interest (e.g., the insert sequence set forth in FIG. 2B) is inserted in the multiple cloning site (MCS), under the control of the TRE. The TRE consists of seven copies of the 42-bp TeTO sequence, and is located just upstream of the minimal immediate early promoter of cytomegalovirus (PminCMV).

### Example 4

This Example demonstrates that in in vitro experiments, when Sophin C expression is induced in breast cancer cell lines, the cells become multinucleate and undergo significant apoptosis, while uninduced control cells remain viable.

Induction of Apoptosis in Small Cell Carcinoma and Breast Cancer Cells by Infection With pRetro-Oncolysin

50,000 Breast tumor cells were infected with approximately 500,000 viral particles in DME+10% FBS containing 4 μg/ml polybrene. After 48 h., the MDA-MB-231 cells were induced with 3 μg of Doxycycline for 6 hours in defined media. Apoptosis was assessed using the FragEL™ apoptotic assay. Uninduced cells are viable and labeled blue when viewed at 10× magnification under a light microscope. Cells expressing oncolysin 1/Sophin C undergo apoptosis as noted by the brownish staining of cells viewed at 10× magnification. Each experiment was performed in duplicates and repeated 3 times. Similar results were obtained when small cell lung carcinoma cells were infected with oncolysin 1/Sophin C producing viral particles.

Tubulin Staining in MDA-MB-231 Human Breast Cancer Cells Expressing Oncolysin

Oncolysin 1-infected MDA-MB-231 tumor cells were induced with 3 μg/ml of Doxycycline. After six hours, the cells were fixed in 10% formaldehyde then stained for tubulin using indirect fluorescent immunochemistry. Control uninduced infected MDA-MB-231 cells showed typical tubulin staining mainly around the nucleus. Induced MDA-MB-231 cells showed stabilized tubulin around multi-nuclei. Notably, the effects are similar to those induced by taxol, a non-receptor-mediated apoptotic agent.

Nuclear Stain of MDA-MB-231 Human Breast Cancer Cell Expressing Oncolysin

Infected cells were stained with H and E without induction with doxycyclin or after induction for six hours. Multinucleation was observed only in induced cells, indicative of the apoptotic phenotype.

### Example 5

This Example Demonstrates that Oncolysin 1/Sophin C Administered in vivo is a an Effective Anti-Tumor Agent.

To evaluate the effectiveness of oncolysin 1/Sophin C against primary tumor growth and metastasis, 1×10⁷ MDA-MB-231 breast cancer cells were injected subcutaneously into the left flank of nude mice. After six weeks the resulting tumors was aseptically dissected out, minced and 1 mm-tumor pieces were transplanted into the right flank of nude mice using a trocar needle. One weeks later, when tumors measured approximately 10 mm, mice were assigned to different experimental groups. One set of 24 animals bearing MDA-MB-231 xenografts were divided into 3 groups that received the following treatments: A first group of 8 animals were injected with pRetro-oncolysin (1×10⁶ viral particles). After one day and weekly thereafter (for 3 weeks) these animals received a weekly injection of the inducing agent Doxycyxlin (1 mg/kg) through the peritoneal cavity. A second group of 8 animals received a weekly injection of Doxycyclin (uninfected tumors) and a third group received a weekly injection of oncolysin protein (10 μg/kg). In another set of experiments, the pRetro-oncolysin was injected into the marrow stroma of tumor bearing mice. After, day one, the animals were treated as above, with weekly injection of Doxycyclin. Tumors were measured once every two days with microcalipers, and tumor volume was calculated as length×width×height×0.5236. Body weight was measured on the day of the injection, 4 days later and weekly thereafter. Four days after the first injection, blood samples were collected from the tail vein using the Unopette™ micro-collection kit. Total leukocyte and platelet counts were determined manually using a hemocytometer. Blood smears stained with the Hema3™ kit were used for assessing absolute numbers of granulocytes and lymphocytes. Treatment-related toxicity was evaluated based on the differences in body weight, liver and kidney marker enzymes, and hematological parameters between treatment groups. 20 weeks after treatment, animals were killed by decapitation under anesthesia. Tumors were dissected, weighed and snap-frozen for caspase enzyme determination. In some cases, the tumors were fixed, and examined histologically. Liver, heart, uterus, ovaries, lungs spinal cord and all long bones were evaluated histologically.

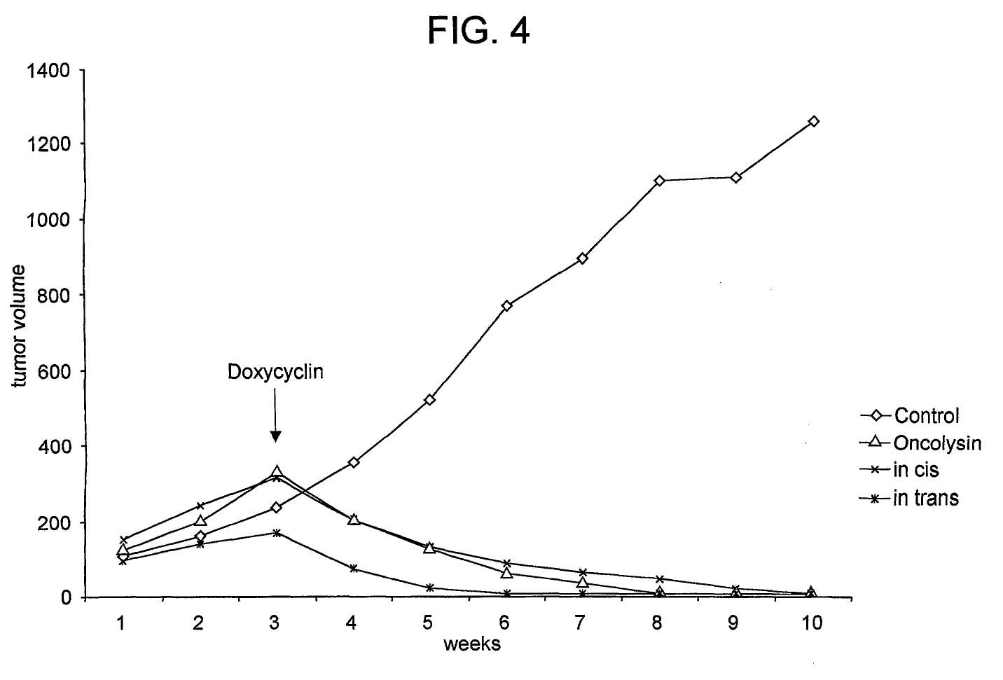

The results shown in FIG. 4 reveal a striking reduction in tumor volume in groups 1 and 3 compared to controls. All treated mice remained healthy after approximately 6 months. In contrast, all control animals either died as a result of their tumors, or were sacrificed as a result of excessive tumor burden. The results demonstrate the effectiveness of Sophin C in vivo in reducing tumor burden and extending viability. In addition they demonstrate the potential for Sophin C to be administered directly or by viral delivery systems.

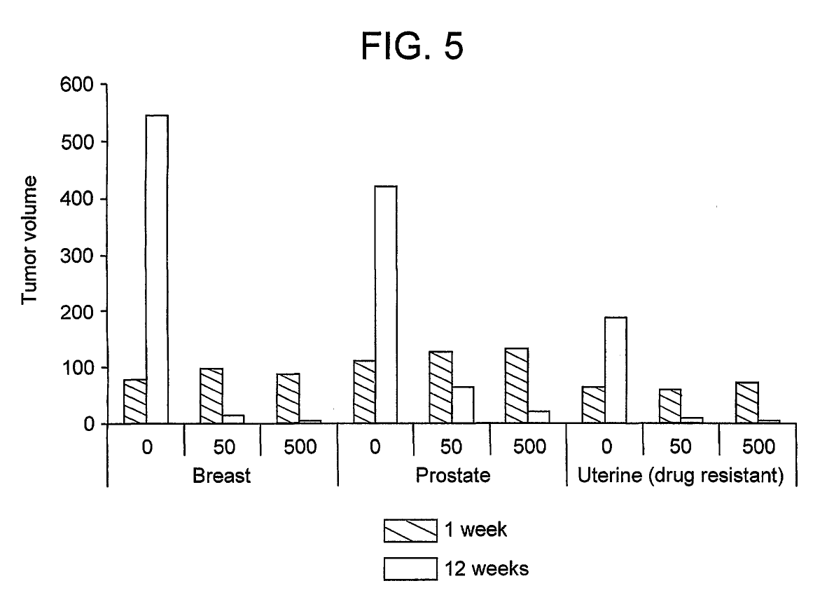

The results shown in FIG. 5 demonstrate experiments to assess efficacy in a broad range of tumor models in addition to dose response studies. The effect of Sophin C on tumor growth was evaluated for three tumor types, breast, prostate and uterine. Results demonstrating a reduction in tumor volume at both 50 and 500 μg/kg protein are shown in FIG. 5.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A biosynthetic oncolytic molecule comprising an apoptotic component and at least one biomodular component, forming a molecule which promotes apoptosis.

2. The oncolytic molecule of claim 1, wherein the apoptotic component is derived from osteopontin.

3. The oncolytic molecule of claim 1, wherein the apoptotic component is a polypeptide.

4. The oncolytic molecule of claim 3, wherein the apoptotic component comprises an amino acid sequence between 5 and 50 amino acid residues in length and is at least 90% identical amino acid residues 147 to 170 sequence of SEQ ID NO: 1.

5. The oncolytic molecule of claim 3, wherein the apoptotic component comprises residues 147 to 170 of SEQ ID NO: 1.

6. The oncolytic molecule of claim 1, wherein the biomodular component is selected from the group consisting of a signal peptide, a linker domain, a golgi processing domain, a heparin binding domain, and a collagen binding domain.

7. A biosynthetic oncolytic molecule comprising an apoptotic component, a first biomodular component and a second biomodular component, forming a molecule which modulates cellular apoptosis.

8. The oncolytic molecule of claim 7, wherein the first and second biomodular components are selected from the group consisting of a signal peptide, a linker domain and a golgi processing domain.

9. The oncolytic molecule of claim 8, further comprising a third biomodular component.

10. The oncolytic molecular of claim 9, wherein the third biomodular component is a heparin binding domain or a collagen binding domain.

11. A biosynthetic oncolytic molecule comprising an apoptotic component, a signal peptide, a linker domain and a golgi processing domain.

12. The oncolytic molecule of claim 11, further comprising a heparin binding domain.

13. The oncolytic molecule of claim 12, further comprising a collagen binding domain.

14. The oncolytic molecule of claim 11, comprising an amino acid sequence sufficiently homologous to the amino acid sequence of SEQ ID NO: 4, wherein the molecule retains an oncolytic activity.

15. The oncolytic molecule of claim 12, comprising an amino acid sequence sufficiently homologous to the amino acid sequence of SEQ ID NO: 5, wherein the molecule retains an oncolytic activity.

16. The oncolytic molecule of claim 13, comprising an amino acid sequence sufficiently homologous to the amino acid sequence of SEQ ID NO: 6, wherein the molecule retains an oncolytic activity

17. The oncolytic molecule of claim 1, wherein the molecule modulates an apoptotic response selected from the group consisting of modulation of chromatin structure, cell viability, and cell lysis.

18. The oncolytic molecule of claim 1, wherein the molecule enhances an apoptotic response.

19. The oncolytic molecule of claim 18, wherein the apoptotic response is cell lysis.

20. An isolated nucleic acid molecule comprising nucleic acid sequences which encode a biosynthetic oncolytic molecule.

21. An expression vector comprising the nucleic acid molecule of claim 20.

22. A host cell comprising the vector of claim 20.

23. A method of producing an oncolytic molecule, comprising culturing the host cell of claim 22 under conditions such that the oncolytic molecule is produced.

24. The method of claim 23, further comprising isolating the oncolytic molecule from the medium or the host cell.

25. A pharmaceutical composition comprising the oncolytic molecule of claim 1, and a pharmaceutically acceptable carrier.

26. A method of modulating an apoptotic response in a cell comprising contacting the cell with an oncolytic molecule of claim 1 such that an apoptotic response is modulated.

27. The method of claim 27, wherein the cell is present within a subject and the oncolytic molecule is administered to the subject.

28. A therapeutic polypeptide comprising the amino acid sequence of SEQ ID NO: 4.

29. A therapeutic polypeptide comprising the amino acid sequence of SEQ ID NO: 5.

30. A therapeutic polypeptide comprising the amino acid sequence of SEQ ID NO: 6.

31. A therapeutic polypeptide comprising the amino acid sequence of SEQ ID NO: 8.

32. An isolated nucleic acid molecule encoding the therapeutic polypeptide of claim 28.

33. An isolated nucleic acid molecule encoding the therapeutic polypeptide of claim 29.

34. An isolated nucleic acid molecule encoding the therapeutic polypeptide of claim 30.

35. An isolated nucleic acid molecule encoding the therapeutic polypeptide of claim 31.

36. An isolated nucleic acid molecule comprising the nucleotide sequence of SEQ ID NO: 7.

37. An isolated nucleic acid molecule comprising nucleotides 1-195 of SEQ ID NO: 7.

38. An isolated nucleic acid molecule comprising nucleotides 1-213 of SEQ ID NO: 7.

39. An expression vector comprising the nucleic acid molecule of any one of claims 32-38.

40. A host cell comprising the vector of claim 39.
